(19) United States
(12) Patent Application Publication
(10) Pub. No.: US 2025/0312603 A1
(43) Pub. Date: Oct. 9, 2025

(54) PERSONALIZED NEURAL-HEALTH BASED STIMULATION

(71) Applicant: Cochlear Limited, Macquarie University, NSW (AU)

(72) Inventors: Naomi Croghan, Centennial, CO (US); Harish Krishnamoorthi, Englewood, CO (US); Sara Ingrid Duran, Denver, CO (US); Christopher Joseph Long, Centennial, CO (US)

(21) Appl. No.: 18/864,629

(22) PCT Filed: May 4, 2023
(86) PCT No.: PCT/IB2023/054668

§ 371 Date: Nov. 11, 2024

Related U.S. Application Data
(60) Provisional application No. 63/342,349, filed on May 16, 2022.

(51) Int. Cl. A61N 1/36; A61N 1/02; G16H 20/30

(52) U.S. Cl. CPC A61N 1/3614; A61N 1/025; A61N 1/36038; A61N 1/36046; G16H 20/30

(57) ABSTRACT

Presented herein are techniques for the determination and use of neural health maps. As used herein, a neural health map refers to a mapping that indicates the neural health of neurons within different regions of a complement of neurons. The neural health indicated in/by the neural health map indicates the ability of a neuron to respond to stimulation. Accordingly, a neural health map can indicate if a particular region of a complement of neurons provides normal response to stimulation, decreased response to stimulation, no response to stimulation (i.e., neuron death), etc.

### BACKGROUND

### Field of the Invention

The present invention relates generally to techniques for determining neuron health and applications thereof for neuron stimulation.

### Related Art

Medical devices have provided a wide range of therapeutic benefits to recipients over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or recipient monitoring for a number of years.

The types of medical devices and the ranges of functions performed thereby have increased over the years. For example, many medical devices, sometimes referred to as “implantable medical devices,” now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### SUMMARY

In some aspects, the techniques described herein relate to a method, including: determining, for each electrode of a plurality of electrodes, a distance between each electrode of the plurality of electrodes and one or more neurons; determining a stimulation threshold for each electrode of the plurality of electrodes to evoke a response in the one or more neurons; correlating the stimulation threshold for each electrode of the plurality of electrodes with the distance between each electrode of the plurality of electrodes and the one or more neurons; and generating a neural health map for the one or more neurons based upon the correlating the stimulation threshold for each electrode of the plurality of electrodes with the distance between each electrode of the plurality of electrodes and the one or more neurons.

According to other aspects, the techniques described herein relate to a method including: determining a neural health map for a plurality of neurons excited by a plurality of electrodes; determining stimulation characteristics for the plurality of neurons based on the neural health map; and stimulating the plurality of neurons via the plurality of electrodes based upon the stimulation characteristics.

According to still other aspects, the techniques described herein relate to a method including: determining a neural health map for a plurality of neurons excited by a plurality of electrodes; determining sound processing characteristics based on the neural health map; and processing an audio signal based upon the sound processing characteristics to generate stimulation signals for the plurality of electrodes.

### BRIEF DESCRIPTION OF THE DRAWINGS

Embodiments of the present invention are described herein in conjunction with the accompanying drawings, in which:

FIG. 1A is a schematic diagram illustrating a cochlear implant system with which aspects of the techniques presented herein can be implemented;

FIG. 1B is a side view of a recipient wearing a sound processing unit of the cochlear implant system of FIG. 1A;

FIG. 1C is a schematic view of components of the cochlear implant system of FIG. 1A;

FIG. 1D is a block diagram of the cochlear implant system of FIG. 1A;

FIG. 2 is a schematic diagram of a first arrangement of electrodes and neurons illustrating the neural health map determination techniques presented herein;

FIG. 3 is a schematic diagram of a second arrangement of electrodes and neurons illustrating the neural health map determination techniques presented herein;

FIG. 4 is a schematic diagram of a third arrangement of electrodes and neurons illustrating the neural health map determination techniques presented herein;

FIG. 5 is a flowchart illustrating a process flow for implementing the neural health map determination techniques presented herein;

FIG. 6 is a schematic diagram illustrating a neural health map determined via the neural health map determination techniques presented herein;

FIG. 7 is a flowchart illustrating a generalized process flow for determining neuron stimulation characteristics based upon a neural health map;

FIG. 8 is a flowchart illustrating a specific example embodiment of a process flow for determining neuron stimulation characteristics based upon a neural health map;

FIG. 9 is a comparison of audio signal and neuron excitation energies to which the neuron stimulation characteristic determination techniques presented herein can be applied;

FIG. 10 is a functional block of an audio signal processing chain to which the audio signal processing characteristics determination techniques presented herein can be applied;

FIG. 11 is a functional block of a warped filter bank to which the audio signal processing characteristics determination techniques presented herein can be applied;

FIG. 12 is a flowchart illustrating a generalized process flow for determining audio signal processing characteristics based upon a neural health map;

FIG. 13 is a schematic diagram illustrating an implantable stimulator system with which aspects of the techniques presented herein can be implemented;

FIG. 14 is a schematic diagram illustrating a vestibular stimulator system with which aspects of the techniques presented herein can be implemented;

FIG. 15 is a schematic diagram illustrating a retinal prosthesis system with which aspects of the techniques presented herein can be implemented; and

FIG. 16 is a schematic diagram illustrating a computing system with which aspects of the techniques presented herein can be implemented.

### DETAILED DESCRIPTION

Presented herein are techniques for the determination and use of neural health maps. As used herein, a neural health map refers to a mapping that indicates the neural health of neurons within different regions of a complement of neurons. The neural health indicated in/by the neural health map indicates the ability of a neuron to respond to stimulation. Accordingly, a neural health map can indicate if a particular region of a complement of neurons provides normal response to stimulation, decreased response to stimulation, no response to stimulation (i.e., neuron death), etc.

Determining the response of a complement of neurons (e.g., a nerve, such as an auditory nerve), to stimulation has been addressed with limited success in conventional systems. Typically, following the surgical implantation of a neural stimulator, such as a cochlear implant, a vestibular stimulator, a retinal prosthesis or others known to the skilled artisan, the stimulator is fitted or customized to conform to the specific recipient demands. This involves the collection and determination of patient-specific parameters such as threshold levels (T levels) and maximum comfort levels (C levels) for each stimulation channel.

One method of interrogating the performance of an implanted neural stimulator and making objective measurements of patient-specific data, such as T and C levels, is to directly measure the response of the nerve to an electrical stimulus. The direct measurement of neural responses, commonly referred to as Electrically-evoked Compound Action Potentials (ECAPs) in the context of cochlear implants, provides an objective measurement of the response of the nerves to electrical stimulus. Following electrical stimulation, the neural response is caused by the superposition of single neural responses at the outside of the axon membranes. The measured neural response is transmitted to an externally-located system, typically via a telemetry system. As a result, the ECAPs are measured from within the cochlea in response to various stimulation signals. The measurements taken to determine whether a neural response or ECAP has occurred are referred to herein by the common vernacular ECAP measurements. The minimal amount of stimulation determined to result in a neural response or ECAP occurrence is referred to herein as the ECAP threshold or, more simply, as the neural response threshold. As described in detail below, the techniques disclosed herein can combine the above-described objective measurements of patient specific data with physical measurement of electrode placement to determine neural health maps for the complement of neurons stimulated by the electrodes of the implanted stimulator.

Merely for ease of description, the techniques presented herein are primarily described with reference to a specific implantable medical device system, namely a cochlear implant system. However, it is to be appreciated that the techniques presented herein can also be partially or fully implemented by other types of implantable medical devices. For example, the techniques presented herein can be implemented by other auditory prosthesis systems that include one or more other types of auditory prostheses, such as middle ear auditory prostheses, bone conduction devices, direct acoustic stimulators, electro-acoustic prostheses, auditory brain stimulators, combinations or variations thereof, etc. The techniques presented herein can also be implemented by dedicated tinnitus therapy devices and tinnitus therapy device systems. In further embodiments, the presented herein can also be implemented by, or used in conjunction with, vestibular devices (e.g., vestibular implants), visual devices (i.e., bionic eyes), sensors, pacemakers, drug delivery systems, defibrillators, functional electrical stimulation devices, catheters, seizure devices (e.g., devices for monitoring and/or treating epileptic events), sleep apnea devices, electroporation devices, etc.

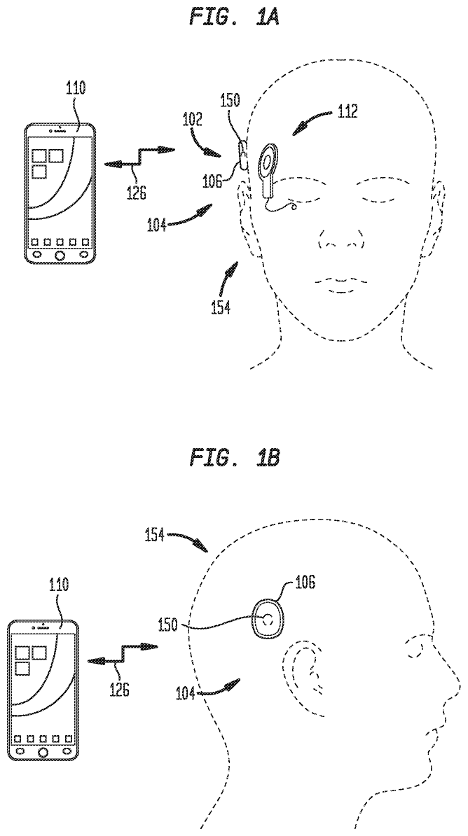

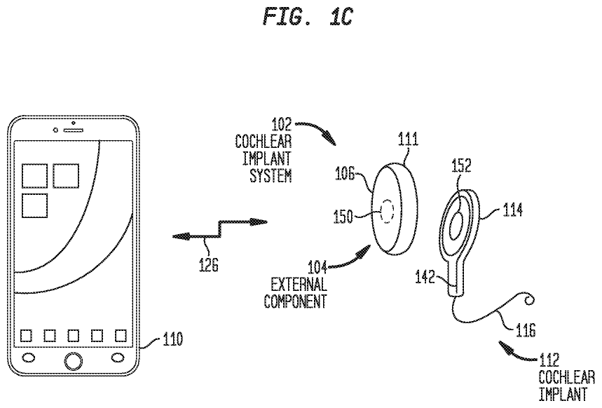

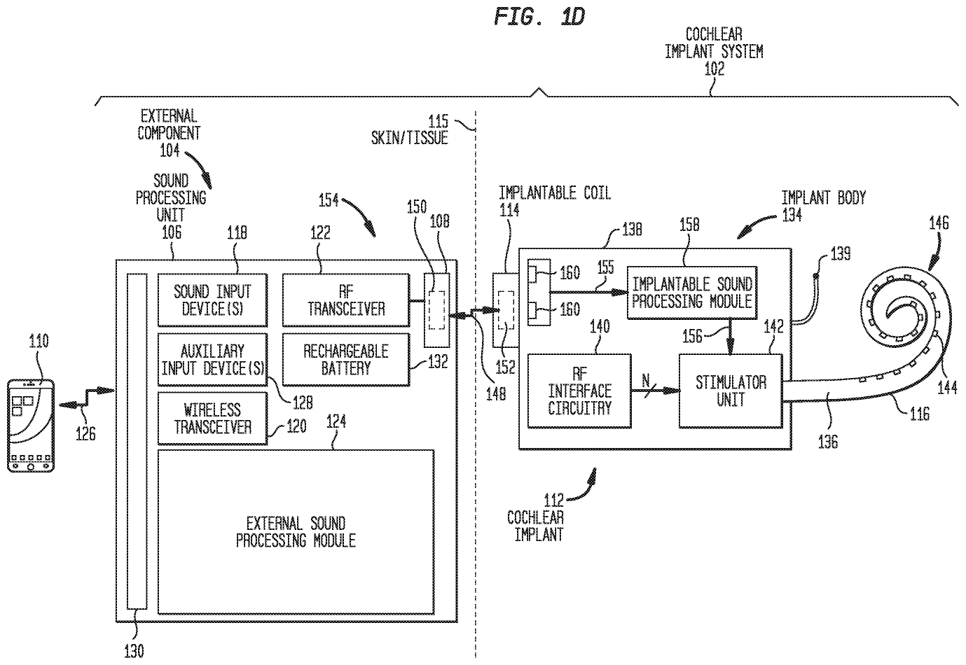

FIGS. 1A-1D illustrates an example cochlear implant system 102 with which aspects of the techniques presented herein can be implemented. The cochlear implant system 102 comprises an external component 104 and an implantable component 112. In the examples of FIGS. 1A-1D, the implantable component is sometimes referred to as a “cochlear implant.” FIG. 1A illustrates the cochlear implant 112 implanted in the head 154 of a recipient, while FIG. 1B is a schematic drawing of the external component 104 worn on the head 154 of the recipient. FIG. 1C is another schematic view of the cochlear implant system 102, while FIG. 1D illustrates further details of the cochlear implant system 102. For ease of description, FIGS. 1A-1D will generally be described together.

Cochlear implant system 102 includes an external component 104 that is configured to be directly or indirectly attached to the body of the recipient and an implantable component 112 configured to be implanted in the recipient. In the examples of FIGS. 1A-1D, the external component 104 comprises a sound processing unit 106, while the cochlear implant 112 includes an implantable coil 114, an implant body 134, and an elongate stimulating assembly 116 configured to be implanted in the recipient's cochlea.

In the example of FIGS. 1A-1D, the sound processing unit 106 is an off-the-ear (OTE) sound processing unit, sometimes referred to herein as an OTE component, which is configured to send data and power to the implantable component 112. In general, an OTE sound processing unit is a component having a generally cylindrically shaped housing 111 and which is configured to be magnetically coupled to the recipient's head (e.g., includes an integrated external magnet 150 configured to be magnetically coupled to an implantable magnet 152 in the implantable component 112). The OTE sound processing unit 106 also includes an integrated external (headpiece) coil 108 that is configured to be inductively coupled to the implantable coil 114.

It is to be appreciated that the OTE sound processing unit 106 is merely illustrative of the external devices that could operate with implantable component 112. For example, in alternative examples, the external component can comprise a behind-the-ear (BTE) sound processing unit or a micro-BTE sound processing unit and a separate external. In general, a BTE sound processing unit comprises a housing that is shaped to be worn on the outer ear of the recipient and is connected to the separate external coil assembly via a cable, where the external coil assembly is configured to be magnetically and inductively coupled to the implantable coil 114. It is also to be appreciated that alternative external components could be located in the recipient's ear canal, worn on the body, etc.

As noted above, the cochlear implant system 102 includes the sound processing unit 106 and the cochlear implant 112. However, as described further below, the cochlear implant 112 can operate independently from the sound processing unit 106, for at least a period, to stimulate the recipient. For example, the cochlear implant 112 can operate in a first general mode, sometimes referred to as an “external hearing mode,” in which the sound processing unit 106 captures sound signals which are then used as the basis for delivering stimulation signals to the recipient. The cochlear implant 112 can also operate in a second general mode, sometimes referred as an “invisible hearing” mode, in which the sound processing unit 106 is unable to provide sound signals to the cochlear implant 112 (e.g., the sound processing unit 106 is not present, the sound processing unit 106 is powered-off, the sound processing unit 106 is malfunctioning, etc.). As such, in the invisible hearing mode, the cochlear implant 112 captures sound signals itself via implantable sound sensors and then uses those sound signals as the basis for delivering stimulation signals to the recipient. Further details regarding operation of the cochlear implant 112 in the external hearing mode are provided below, followed by details regarding operation of the cochlear implant 112 in the invisible hearing mode. It is to be appreciated that reference to the external hearing mode and the invisible hearing mode is merely illustrative and that the cochlear implant 112 could also operate in alternative modes.

In FIGS. 1A and 1C, the cochlear implant system 102 is shown with an external device 110, configured to implement aspects of the techniques presented. The external device 110 is a computing device, such as a computer (e.g., laptop, desktop, tablet), a mobile phone, remote control unit, etc. As described further below, the external device 110 comprises a telephone enhancement module that, as described further below, is configured to implement aspects of the auditory rehabilitation techniques presented herein for independent telephone usage. The external device 110 and the cochlear implant system 102 (e.g., OTE sound processing unit 106 or the cochlear implant 112) wirelessly communicate via a bi-directional communication link 126. The bi-directional communication link 126 can comprise, for example, a short-range communication, such as Bluetooth link, Bluetooth Low Energy (BLE) link, a proprietary link, etc.

Returning to the example of FIGS. 1A-1D, the OTE sound processing unit 106 comprises one or more input devices configured to receive input signals (e.g., sound or data signals). The one or more input devices include one or more sound input devices 118 (e.g., one or more external microphones, audio input ports, telecoils, etc.), one or more auxiliary input devices 128 (e.g., audio ports, such as a Direct Audio Input (DAI), data ports, such as a Universal Serial Bus (USB) port, cable port, etc.), and a wireless transmitter/receiver (transceiver) 120 (e.g., for communication with the external device 110). However, it is to be appreciated that one or more input devices can include additional types of input devices and/or less input devices (e.g., the wireless short range radio transceiver 120 and/or one or more auxiliary input devices 128 could be omitted).

The OTE sound processing unit 106 also comprises the external coil 108, a charging coil 130, a closely-coupled transmitter/receiver (RF transceiver) 122, sometimes referred to as or radio-frequency (RF) transceiver 122, at least one rechargeable battery 132, and an external sound processing module 124. The external sound processing module 124 can comprise, for example, one or more processors and a memory device (memory) that includes sound processing logic. The memory device can comprise any one or more of: Non-Volatile Memory (NVM), Ferroelectric Random Access Memory (FRAM), read only memory (ROM), random access memory (RAM), magnetic disk storage media devices, optical storage media devices, flash memory devices, electrical, optical, or other physical/tangible memory storage devices. The one or more processors are, for example, microprocessors or microcontrollers that execute instructions for the sound processing logic stored in memory device.

The implantable component 112 comprises an implant body (main module) 134, a lead region 136, and the intra-cochlear stimulating assembly 116, all configured to be implanted under the skin/tissue (tissue) 115 of the recipient. The implant body 134 generally comprises a hermetically-sealed housing 138 in which RF interface circuitry 140 and a stimulator unit 142 are disposed. The implant body 134 also includes the internal/implantable coil 114 that is generally external to the housing 138, but which is connected to the RF interface circuitry 140 via a hermetic feedthrough (not shown in FIG. 1D).

As noted, stimulating assembly 116 is configured to be at least partially implanted in the recipient's cochlea. Stimulating assembly 116 includes a plurality of longitudinally spaced intra-cochlear electrical stimulating contacts (electrodes) 144 that collectively form a contact or electrode array 146 for delivery of electrical stimulation (current) to the recipient's cochlea.

Stimulating assembly 116 extends through an opening in the recipient's cochlea (e.g., cochleostomy, the round window, etc.) and has a proximal end connected to stimulator unit 142 via lead region 136 and a hermetic feedthrough (not shown in FIG. 1D). Lead region 136 includes a plurality of conductors (wires) that electrically couple the electrodes 144 to the stimulator unit 142. The implantable component 112 also includes an electrode outside of the cochlea, sometimes referred to as the extra-cochlear electrode (ECE) 139.

As noted, the cochlear implant system 102 includes the external coil 108 and the implantable coil 114. The external magnet 152 is fixed relative to the external coil 108 and the implantable magnet 152 is fixed relative to the implantable coil 114. The magnets fixed relative to the external coil 108 and the implantable coil 114 facilitate the operational alignment of the external coil 108 with the implantable coil 114. This operational alignment of the coils enables the external component 104 to transmit data and power to the implantable component 112 via a closely-coupled wireless link 148 formed between the external coil 108 with the implantable coil 114. In certain examples, the closely-coupled wireless link 148 is a radio frequency (RF) link. However, various other types of energy transfer, such as infrared (IR), electromagnetic, capacitive and inductive transfer, can be used to transfer the power and/or data from an external component to an implantable component and, as such, FIG. 1D illustrates only one example arrangement.

As noted above, sound processing unit 106 includes the external sound processing module 124. The external sound processing module 124 is configured to convert received input signals (received at one or more of the input devices) into output signals for use in stimulating a first ear of a recipient (i.e., the external sound processing module 124 is configured to perform sound processing on input signals received at the sound processing unit 106). Stated differently, the one or more processors in the external sound processing module 124 are configured to execute sound processing logic in memory to convert the received input signals into output signals that represent electrical stimulation for delivery to the recipient.

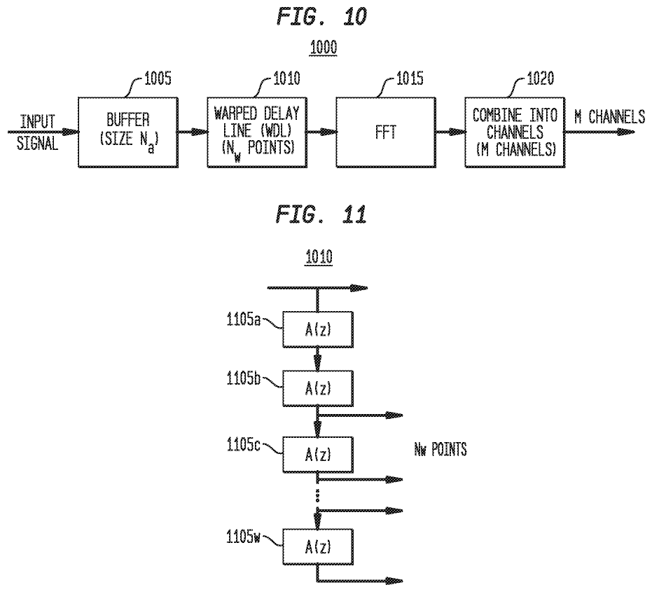

As explained in detail below with reference to FIGS. 10 and 11, specific example embodiments of external sound processing module 124 can be configured with a warped filter bank via which applications of the neural health maps are implemented in cochlear implant system 102.

As noted, FIG. 1D illustrates an embodiment in which the external sound processing module 124 in the sound processing unit 106 generates the output signals. In an alternative embodiment, the sound processing unit 106 can send less processed information (e.g., audio data) to the implantable component 112 and the sound processing operations (e.g., conversion of sounds to output signals) can be performed by a processor within the implantable component 112.

Returning to the specific example of FIG. 1D, the output signals are provided to the RF transceiver 122, which transcutaneously transfers the output signals (e.g., in an encoded manner) to the implantable component 112 via external coil 108 and implantable coil 114. That is, the output signals are received at the RF interface circuitry 140 via implantable coil 114 and provided to the stimulator unit 142. The stimulator unit 142 is configured to utilize the output signals to generate electrical stimulation signals (e.g., current signals) for delivery to the recipient's cochlea. In this way, cochlear implant system 102 electrically stimulates the recipient's auditory nerve cells, bypassing absent or defective hair cells that normally transduce acoustic vibrations into neural activity, in a manner that causes the recipient to perceive one or more components of the received sound signals.

As detailed above, in the external hearing mode the cochlear implant 112 receives processed sound signals from the sound processing unit 106. However, in the invisible hearing mode, the cochlear implant 112 is configured to capture and process sound signals for use in electrically stimulating the recipient's auditory nerve cells. In particular, as shown in FIG. 1D, the cochlear implant 112 includes a plurality of implantable sound sensors 160 and an implantable sound processing module 158. Similar to the external sound processing module 124, the implantable sound processing module 158 can comprise, for example, one or more processors and a memory device (memory) that includes sound processing logic. The memory device can comprise any one or more of: Non-Volatile Memory (NVM), Ferroelectric Random Access Memory (FRAM), read only memory (ROM), random access memory (RAM), magnetic disk storage media devices, optical storage media devices, flash memory devices, electrical, optical, or other physical/tangible memory storage devices. The one or more processors are, for example, microprocessors or microcontrollers that execute instructions for the sound processing logic stored in memory device.

Specific example embodiments of implantable sound processing module 158 can be configured with a warped filter bank via which applications of the neural health maps are implemented in cochlear implant system 102, as described in detail with reference to FIGS. 10 and 11 below.

In the invisible hearing mode, the implantable sound sensors 160 are configured to detect/capture signals (e.g., acoustic sound signals, vibrations, etc.), which are provided to the implantable sound processing module 158. The implantable sound processing module 158 is configured to convert received input signals (received at one or more of the implantable sound sensors 160) into output signals for use in stimulating the first ear of a recipient (i.e., the processing module 158 is configured to perform sound processing operations). Stated differently, the one or more processors in implantable sound processing module 158 are configured to execute sound processing logic in memory to convert the received input signals into output signals 156 that are provided to the stimulator unit 142. The stimulator unit 142 is configured to utilize the output signals 156 to generate electrical stimulation signals (e.g., current signals) for delivery to the recipient's cochlea, thereby bypassing the absent or defective hair cells that normally transduce acoustic vibrations into neural activity.

It is to be appreciated that the above description of the so-called external hearing mode and the so-called invisible hearing mode are merely illustrative and that the cochlear implant system 102 could operate differently in different embodiments. For example, in one alternative implementation of the external hearing mode, the cochlear implant 112 could use signals captured by the sound input devices 118 and the implantable sound sensors 160 in generating stimulation signals for delivery to the recipient.

As noted, above presented herein are techniques for the determination and use of neural health maps from, as explained in detail below, objective measurements obtained/captured via components of a neural stimulator, such as electrode assembly 146 of FIG. 1D. In certain embodiments, these objective measurements are combined with physical measurements of electrode placement to determine the neural health of the neurons stimulated by the neural stimulator.

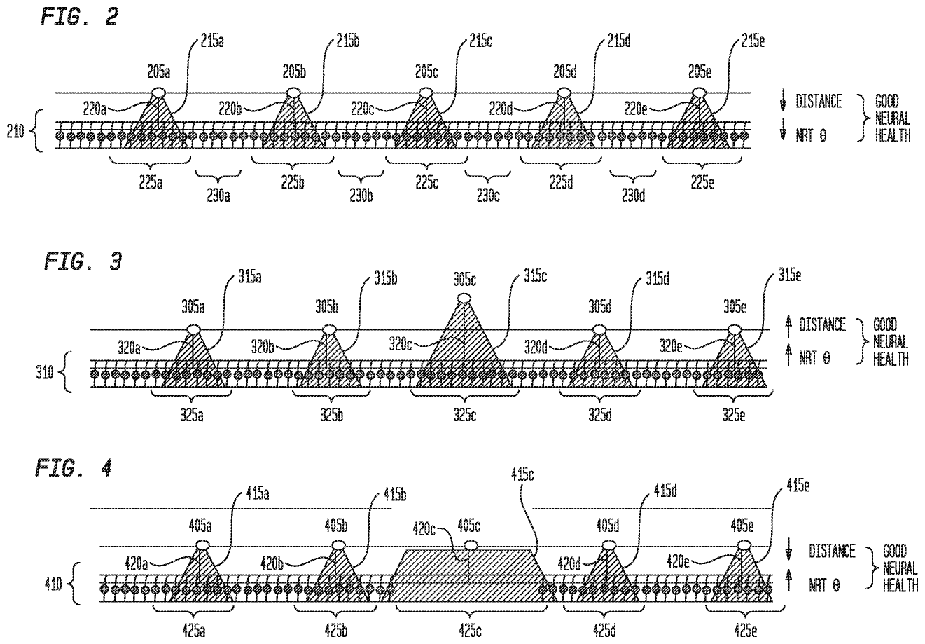

With reference now made to FIG. 2, depicted therein are a series of electrodes 205a-e arranged relative to a complement of neurons 210, such as the neurons arranged about the modiolus of the cochlea. As explained below, the distances 220a-e between electrodes 205a-e and the neurons 210 are obtained from a physical measurement of electrode placement, such as Computed Tomography (CT), x-ray or magnetic resonance imaging of the electrodes.

Additional techniques for determining electrode placement can include Electrode Voltage Tomography (EVT) techniques. According to certain EVT techniques, EVT measurements include a plurality of sequential measurement sets that each involve the delivery of current between one of the intra-cochlea electrodes of a cochlear implant (e.g., electrodes 144 of FIG. 1D) and the extra-cochlear electrode of the implant (e.g., extra-cochlear electrode 139 of FIG. 1D). With each delivery of current between the one of the intra-cochlea electrodes and the extra-cochlear electrode, voltage measurements between the extra-cochlear electrode and each of the other intra-cochlea electrodes are obtained. This process can be repeated for one or more (or all) of the other intra-cochlea electrodes as the current delivery electrode. Based upon the obtained measurements, the placement or distance of the electrodes relative to the neurons can be determined. The EVT measurements can be stored in a matrix known as a trans-impedance matrix (TIM). The values in the TIM characterize the electrical characteristics of the electrodes and tissue surrounding the electrode assembly.

In order to determine the TIM for each electrode of the stimulating assembly (e.g., electrode assembly 146 of FIG. 1D), current is applied to a first electrode at a known current level, i, and the resulting voltage, v, is recorded/measured at all of the other electrodes. The trans-impedances, z, denoted as below in Equation 1, are calculated for each combination of stimulating electrode and recording electrode:

$${z}_{i , j} = {v}_{i} / {i}_{j} , \qquad ( 1 )$$

where vᵢ is the voltage measured at a recording electrode, iⱼ is the current delivered at the stimulating electrode and z_{i,j} is the transimpedance measured there between. The trans-impedances calculated for all pairs of electrodes are combined to form a full trans-impedance matrix (Z). For a 22-electrode assembly, the result is a 22×22 trans-impedance matrix. The values stored in the TIM can be used to determine the location of the electrodes relative to the neurons of the cochlea.

Regardless of the method used to determine the distances 220a-e, the techniques of the present disclosure correlate the distances 220a-e with the stimulation signals (stimulations) 215a-e necessary to evoke a response of the complement of neurons 210 in regions 225a-e, respectively. For the purposes of the present disclosure, the illustrated magnitudes of the stimulation signals 215a-e, which are represented by the shaded regions, are generally indicative of the level/threshold of stimulation needed to evoke a response in the complement of neurons 210 within regions 225a-e, respectively.

In the example of FIG. 2, the correlation of the distances 220a-e to the stimulation signals 215a-e can be used to determine neural health within regions 225a-e, respectively. More specifically FIG. 2 illustrates that electrodes 205a-e are all relatively equally spaced from the complement of neurons 210. Accordingly, distances 220a-e are all of a similar magnitude. FIG. 2 also illustrates the strength of the stimulation signals 215a-e used to evoke a response, which are also relatively equal as indicated by the illustrated magnitudes of the stimulation signals 215a-e. As such, because the distances 220a-e between electrodes 205a-e and complement of neurons 210, respectively, are relatively the same, and the magnitude of the stimulation signals 215a-e needed to evoke a response in the complement of neurons 210 (i.e., the stimulation thresholds) in regions 225a-e are relatively the same, it can be inferred that regions 225a-e share a similar level of neuron health. Moreover, because both the estimated distances 220a-e from the electrodes 205a-e to the complement of neurons 210 and the stimulation signals 215a-e are low it is determined that all neurons within regions 225a-e have a good level of neural health. Accordingly, a neural health map can be determined for regions 225a-e in which all of the regions have a normal level of neural health.

Turning to FIG. 3, illustrated therein are electrodes 305a-e and a complement of neurons 310, similar to electrodes 205a-e and complement of neurons 210 of FIG. 2. Similar to FIG. 2, the distances 320a-e between electrodes 305a-e and the neurons 310 are obtained from a physical measurement. The illustrated magnitudes of the stimulation signals 315a-e, which are represented by the shaded regions, are generally indicative of the level of stimulation needed to evoke a response in the complement of neurons 310 within regions 325a-e, respectively.

As shown, the distances 320a, 320b, 320d and 320e between electrodes 305a, 305b, 305d and 305e and complement of neurons 310 are substantially the same, but electrode 305c is substantially further from complement of neurons 310. In addition, the magnitude of the stimulation signal 315c that is necessary to evoke a response in region 325c is similarly larger than the magnitudes of the stimulation signals 315a, 315b, 315d and 315e. The increased magnitude of stimulation 315c is not, however, an indication of poor health for the neurons arranged within region 325c. Instead, by correlating the distance and stimulation level/threshold, it is determined that electrode 305c would require increased stimulation to evoke a response in region 325c because distance 320c is greater than distances 320a, 320b, 320d and 320e, not because of decreased neural health within region 325c. Accordingly, a neural health map can be determined for regions 325a-e in which all of the regions have a normal level of neural health.

Illustrated in FIGS. 2 and 3 is a monotonic relationship between the distance from electrodes 215a-e, 305a-e and complements of neurons 210, 310 and the stimulation signals 215a-e, 315a-e needed to evoke a response in regions 225a-e, 325a-e. As the distance between electrodes 215a-e, 305a-e and complements of neurons 210, 310 decreases so does the magnitude of stimulation needed to evoke a response. As the distances 220a-e, 320a-e between electrodes 215a-e, 305a-e and regions 225a-e, 325a-e increase so too does the magnitude of stimulation needed to evoke a response. Accordingly, the large stimulation 315c associated with electrode 305c is not indicative of poor neuron health within region 325c because distance 320c is also correspondingly larger. Turning to FIG. 4, the large stimulation 415c of electrode 405c, on the other hand, is indicative of poor neuron health.

More specifically, FIG. 4 illustrates electrodes 405a-e and a complement of neurons 410, similar to electrodes 205a-e and complement of neurons 210 of FIG. 2. Similar to FIG. 2, the distances 420a-e between electrodes 405a-e and the neurons 410 are obtained from a physical measurement. The illustrated magnitude of the stimulation signals 415a-e, which are represented by the shaded regions, are generally indicative of the level of stimulation needed to evoke a response in the complement of neurons 410 within regions 425a-e, respectively.

Stimulation signal 415c is associated with a larger magnitude of stimulation (as indicated by the larger magnitude of shaded region 415c) to evoke a response in region 425c of complement of neurons 410. Because distance 420c is not appreciably larger than distances 420a, 420b, 420d and 420e, but the magnitude of stimulation signal 415c is appreciably greater than that of stimulation signals 415a, 415b, 415d and 415e, the magnitude of stimulation signal 415c is, in fact, indicative of poor neuron health within region 425c. Similarly, if stimulation signal 415c is increased without any detected response from region 425c, this can serve as an indication of neuron death within region 425c. Accordingly, a neural health map can be determined for regions 425a-e in which regions 425a, 425b, 425d and 425e have a normal level of neural health and region 425c has a poor level of neural health.

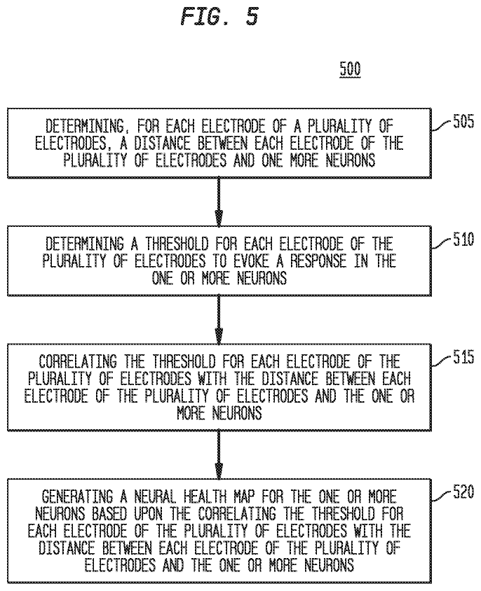

With reference now made to FIG. 5, depicted therein is a flowchart 500 illustrating a generalized process flow for determining a neural health map according to the techniques disclosed herein. Flowchart 500 begins in operation 505 in which a distance is determined from each electrode of a plurality of electrodes to one or more neurons, where the plurality of electrodes can include all, or only a subset of electrodes (e.g., a group of two electrodes, a group of three electrodes, etc.), implanted in a recipient. Accordingly, operation 505 can be the process by which distances 220a-e, 320a-e and/or 420a-e are determined. For example, CT imaging, x-ray imaging or magnetic resonance imaging can be used to determine the location of each electrode of an electrode array (such as electrode assembly 146 of FIG. 1D) relative to the neurons that the electrodes are intended to stimulate. Other methods of determining the location of the one more electrodes can also be used, such as the EVT/TIM techniques described above.

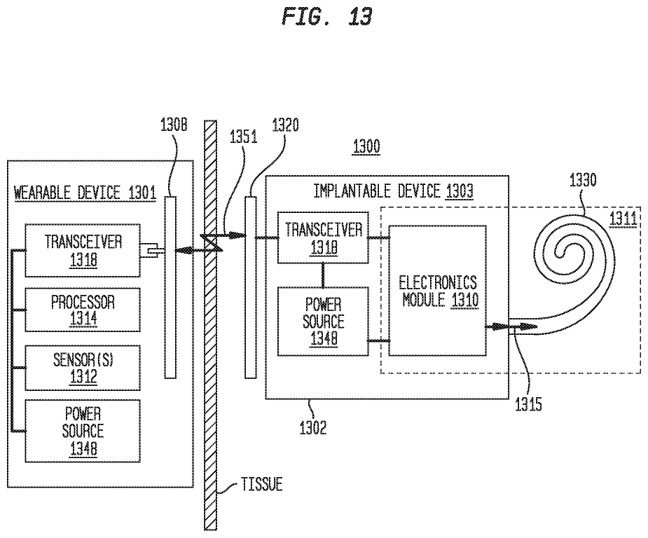

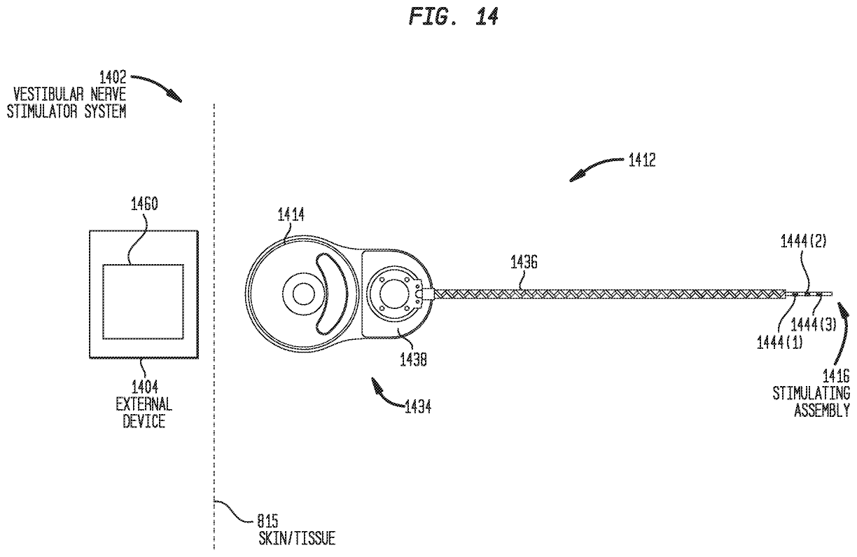

Next, in operation 510, a threshold to evoke a response in the one or more neurons is determined for each electrode of the plurality of electrodes. For example, operation 510 can include the process by which the magnitudes of stimulation signals 215a-e, 315a-e and/or 415a-e are determined. For cochlear implant recipients, operation 510 can be embodied as the determination of a Neural response threshold for each electrode for the plurality of electrodes of the implant. Specifically, telemetry software can be used in conjunction with the plurality of electrodes to calculate the Neural response threshold for each of the electrodes. For other types of neurons, such as those described below with reference to FIGS. 13-15, operation 510 can determine a different type of threshold, so long as the determined threshold is that which relates the level of stimulation to evoking a response in the one or more neurons.

According to one specific example in which Neural response thresholds are calculated in operation 510, software can be utilized that has built-in algorithms that mark the negative and positive peaks of the ECAP from the auditory nerve in a cochlear implant recipient, that calculates the peak-to-peak amplitude difference, and then plots that difference as a function of current level. This plot is called an amplitude growth function or an input-output function. The Neural response threshold is derived from a linear regression applied to the data points in the growth function.

Monopolar stimulation can be used in operation 510 to determine the Neural response threshold for regions of neurons. Though, the techniques disclosed herein are not limited to just monopolar stimulation. Bipolar, tripolar, or focused multipolar stimulation techniques can be used to generate more focused electric fields. Accordingly, bipolar, tripolar, or focused multipolar stimulation techniques can be used to generate more detailed neural health maps.

To determine the neural health for regions between electrodes, virtual channel stimulation techniques can be used. According to such techniques, the absolute and relative proportion of electrical current for a virtual channel (e.g., a pair of adjacent electrodes activated simultaneously) can be adjusted to produce an inter-electrode place of excitation in the cochlea. Turning briefly to FIG. 2, virtual channel techniques can be leveraged to determine the neural health of regions 230a-d.

Returning to FIG. 5, in operation 515, the threshold for each electrode of the plurality of electrodes is correlated with the distance between each electrode of the plurality of electrodes and the one or more neurons. For example, as described above with reference to FIGS. 2-4, the relationship of the distance from an electrode to a neuron and the amount of stimulation required to excite the neuron can be indicative of neural health. Correlating the distance between the electrode and the neurons and the threshold allows for a determination of the neural health of the neurons stimulated by each of the plurality of electrodes.

Finally, in operation 520, a neural health map is generated for the one or more neurons based upon the correlation that took place in operation 515. Using FIGS. 2-4 as an example, a neural health map for regions 225a-e, 325a-e and 425a-e would indicate good neural health for regions 225a-e, 325a-e, 425a, 425b, 425d and 425e, and poor neural health or neuron death within region 425c.

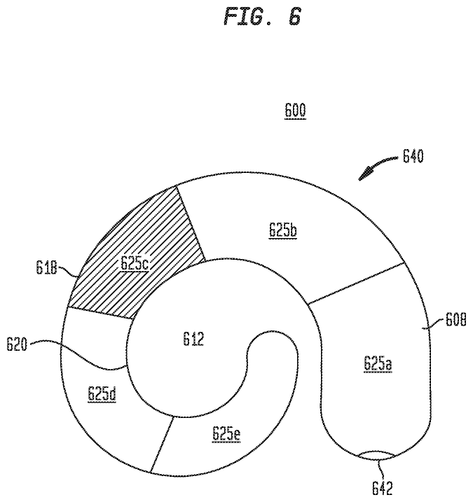

With reference now made to FIG. 6, depicted therein is a neural health map 600 of a cochlea 640, the neural health of which has been mapped according to the techniques disclosed herein. Illustrated in FIG. 6 are the modiolar wall 620 (e.g., the wall of the scala tympani 608 adjacent the modiolus 612) and the lateral wall 618 (e.g., the wall of the scala tympani 608 positioned opposite to the modiolus 612). Also shown in FIG. 6 is a cochlea opening 642 which can be, for example, a natural opening (e.g., the round window) or a surgical opening (e.g., a cochleostomy). Cochlea 640 includes a mapping of its neural health in the form of regions 625a-e. As illustrated through its shading, region 625c has been mapped as having poor neural health, while regions 625a, 625b, 625d and 625e have been mapped as having good neural health.

Neural health map 600 will permit the control of a stimulation unit, such as stimulation unit 142 of FIG. 1D, and an electrode array, such as electrode array 146 of FIG. 1D, of an implantable medical device to address the specific neural health needs of cochlea 640. For example, through the use of neural health map 600, as well as additional information, such as a TIM, stimulation parameters can be adjusted to compensate for various limitations in the electrode-neuron interface. The stimulation parameters adjusted can include, for example, the degree of focusing for focused multipolar stimulation (also referred to herein as the defocusing index (DI)) and/or the assumed spread of excitation. Such techniques can be used to define other stimulation parameters such as current steering, a number of active electrodes, a stimulation rate, and others known to the skilled artisan. A selection process for these parameters can include objective metrics which could be conducted intraoperatively for a personalized first-fit map.

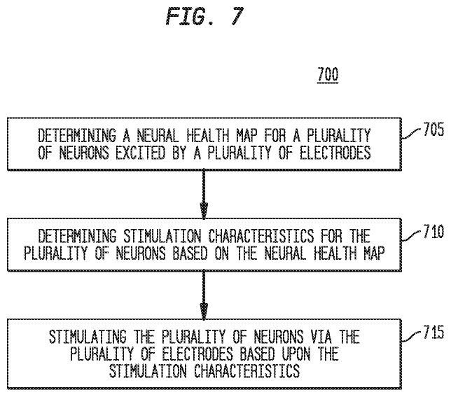

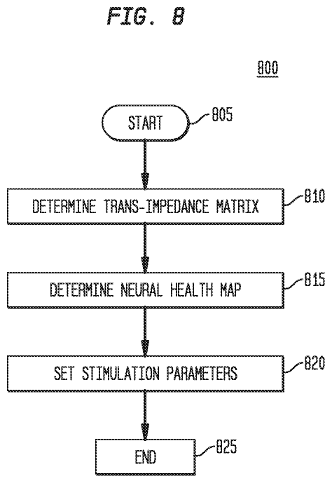

With reference now made to FIG. 7, depicted therein is a flowchart 700 applying a neural health map, such as the neural health maps determined as described above with reference to FIGS. 2-6, to stimulation characteristics for an implanted medical device. Flowchart 700 begins in operation 705 in which a neural health map is determined for a plurality of neurons excited by a plurality of electrodes (e.g., all, or only a subset of electrodes implanted in a recipient). According to specific example embodiments, the determination of operation 705 can be based upon the correlation of the stimulation necessary to evoke a response in an electrode with the distance between the electrode and the neurons. According to other example embodiments, the neural health map of operation 705 can be determined through other techniques known to the skilled artisan. For example, neural health can be determined through other techniques, such as ECAP amplitude growth function (AGF) slopes, the effect of changing inter-phase gap (IPG) on the AGF curve, spread of excitation (SOE) measures using panoramic ECAP (PECAP), the effect of changing polarity on the stimulation of the AGF curve (also referred to as the “polarity effect”), and the effect of measured latency of the neural response, among others known to the skilled artisan. Because the EVT and/or the TIM techniques described above characterize the electrical characteristics of the electrodes and tissue surrounding the electrode assembly, a neural health map can be determined by creating channels using EVT measurements and/or a TIM determined from EVT measurements. These methods can determine neuron health independent of electrode distance and can not require knowledge of electrode placement.

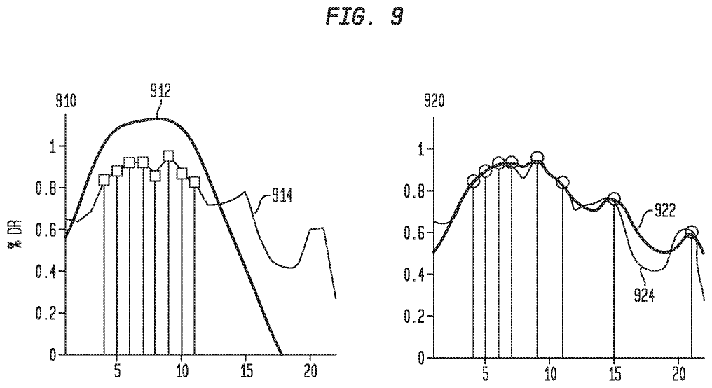

In operation 710 stimulation characteristics for the plurality of neurons are determined based on the neural health map. The determination of the stimulation characteristics can include determining stimulation characteristics for each of a plurality of regions associated with the plurality of neurons. The characteristics determined in operation 710 can include determining which regions associated with the plurality of neurons should be stimulated, the type of stimulation (e.g., monopolar, bipolar, tripolar, etc.), the number or type of stimulation channels (e.g., actual vs. virtual stimulation channels), a degree of focusing for multipolar stimulation, a spread of excitation of the stimulation, a level of stimulation, and other characteristics understood by the skilled artisan. Operation 710 can also incorporate additional information, such as a TIM, in determining the stimulation characteristics. An example that utilizes a TIM is described below with reference to FIG. 8.

Finally, in operation 715, the plurality of neurons are stimulated via the electrodes based upon the stimulation characteristics determined in operation 710.

A specific example of determining stimulation parameters using a neural health map will now be described with reference to flowchart 800 of FIG. 8. The specific example of flowchart 800 uses a TIM in conjunction with a neural health map to set stimulation parameters for electrodes used in the stimulation of neurons. Flowchart 800 begins in operation 805 and proceeds to operation 810 where a TIM is determined for the electrodes to be used in the stimulation.

As discussed above, a TIM refers to a matrix of electrical measurements that can be used to infer states of the electrode array insertion into the cochlea, such as positioning, integrity of the electrode array and anomalous events. In other words, the TIM characterizes the electrical characteristics of the electrodes and tissue surrounding the electrode assembly. For example, a TIM can be used to define a stimulation channel, such as controlling the width of stimulations 215a-e, 315a-e and 415a-e of FIGS. 2-4, respectively. Through the use of a TIM, regions 215a-e, 315a-e and 425a-e of FIGS. 2-4, respectively, can be targeted in a broad or narrow manner depending on how focused the channels are determined to be. Other uses of the TIM can include determining electrodes experiencing “tip foldover” (i.e., when an electrode array folds over on itself), as well as in inferring electrode positioning using EVT techniques.

Once the TIM is determined in operation 810, flowchart 800 proceeds to operation 815 in which a neural health map is determined. Operation 815 can be embodied by a process as described above with reference to FIGS. 2-7. Specifically, the position of the electrodes relative to the neurons can be determined from imaging or another physical measurement. The position of the electrodes can also be determined from the TIM determined in operation 810.

Once the position of the electrodes is determined, Neural response thresholds for each of the electrodes can be determined. The Neural response threshold for each electrode can then be correlated with its known distance to determine a neural health map for the neurons to be stimulated. According to other example embodiments, the neural health map of operation 815 can be determined through other techniques known to the skilled artisan. For example, ECAP AGF slopes, the effect of changing IPG on the AGF curve, and SOE measures using panoramic PECAP can be used to determine a neural health map. These methods can determine neuron health independent of electrode distance and may not require knowledge of electrode placement.

EVT techniques and/or a TIM can also be used in the determination of the neural health map, as described above. Furthermore, depending on how the neural health map is determined, operation 815 can precede operation 810 without deviating from the techniques disclosed herein. For example, if operation 815 is embodied by the process illustrated in flowchart 500 of FIG. 5, the neural health map can be determined either prior or subsequent to the determination of the TIM without deviating from the techniques disclosed herein.

Once the TIM and neural health map are determined, stimulation parameters are set for the stimulation electrodes in operation 820, and flowchart 800 ends in operation 825.

An example of operation 820 can include setting stimulation parameters for channels with good neural survival. Channels in regions with good estimated neural survival, indicated by a good neural health in the neural health map, can use a greater degree of focusing. The parameters for this greater degree of focusing can be determined for the TIM. Accordingly, the greater degree of focusing is based on both the neural health map (which identifies the regions that receive the greater degree of focusing) and the TIM (from which the specific stimulation parameters that will result in the greater degree of focusing are determined). According to other examples, if a channel is assumed to have good neural survival but has a larger electrode-to-modiolus distance, the amplitude of the current of the stimulation will be increased on the focused channel in accordance with the increase in Neural response threshold. The channel may only be de-focused if necessary due to compliance. In contrast, a channel with poor neural survival, indicated by poor neural health in the neural health map, can benefit from being de-focused (i.e., provide more monopolar stimulation) to ensure a sufficient number of nerve fibers are recruited. Accordingly, the neural health map identifies the regions that receive the de-focused stimulation and the TIM determines the specific stimulation parameters that will result in the de-focused stimulation.

According to other examples of operation 820, the number and spacing of stimulation sites from within the electrode array can also be set as a function of electrode-to-modiolus distance and DI, under the assumption that greater distance and less focusing should result in wider spread of excitation. The assumed spread of excitation can be set on a per-channel basis or using an average across channels for a simplified solution. For example, illustrated in FIG. 9 is a graph 910 which illustrates the neural excitation pattern 912 based upon a received audio signal 914. Specifically, graph 910 illustrates regions of the cochlea on the x-axis that correspond to particular frequencies and the level of energy at those frequencies on the y-axis. Excitation graph 912 illustrates how energy would be distributed across the regions of the cochlea if the stimulation signals were made directly proportional to the energy in audio signal 914. As illustrated, excitation graph 912 does not match audio signal graph 914 due to clustering of stimulation sites causing excitation across overlapping regions. Excitation graph 912 has a loss of fidelity relative to audio signal 914, which can result in a corresponding loss of fidelity in the signals perceived by the cochlear implant recipient.

Graph 920 similarly illustrates the neural excitation pattern 922 based upon a received audio signal 924. Unlike graph 910, excitation graph 922 accounts for the assumed spread of excitation by spacing out stimulation sites, resulting in improved fidelity in excitation graph 922 compared with excitation graph 912, which can be accompanied by a corresponding increase in fidelity in the signals perceived by the cochlear implant recipient. The techniques which select the stimulation energies and regions that result in excitation graph 922 distribute energy across regions of the cochlea. Yet, if the distribution of energy is into regions of poor neural health, the benefits of these techniques may not be realized. Accordingly, a neural health map can be used to better determine the stimulation regions and energies to provide improved fidelity for the cochlear implant recipient.

The neural health maps can also be utilized for purposes other than setting stimulation parameters. For example, the sound processing module of a cochlear implant, such as external sound processing module 124 or implantable sound processing module 158, both of FIG. 1D, can include fast Fourier transform (FFT) filter banks. There is a limited resolution for these transforms. As explained in detail below with reference to FIGS. 10 and 11, a neural health map can be used in conjunction with a warped delay line to tailor the resolution of the FFTs to specific frequencies for individual recipients.

Illustrated in FIG. 10 is a processing chain 1000 incorporated into a signal processor, such as external sound processing module 124 or implantable sound processing module 158, both of FIG. 1D, which receives an input audio signal. Processing chain 1000 includes buffer block 1005, warped delay line (WDL) block 1010, fast Fourier transform (FFT) block 1015, and combine-in-channel (CIC) block 1020. The WDL block 1010 includes a cascade of N_{W}−1 first order all-pass filter sections, as shown through all pass filter sections 1105a-w of FIG. 11. The all-pass sections 1105a-w have a transfer function, A(z), of the following form:

$$A ( z ) = \frac{- λ + {z}^{- 1}}{1 - λ {z}^{- 1}} \qquad ( 2 )$$

where λ is the warping coefficient whose value is between −1<λ<1, and z is a complex variable. The parameter λ is responsible for frequency warping. Specifically, by appropriately selecting the value for λ, the resolution can be improved for certain frequencies of the input audio signal. When a single global λ is used for the entire WDL block 1010, the changes in lambda will increase resolution for higher frequencies to the detriment of lower frequencies, and vice versa, depending on the sign of λ. If a respective local λ is used for each of all pass filter sections 1105a-w, the frequency resolution can be tailored to specific frequency regions/bands.

The use of a neural health map, like those discussed above with reference to FIG. 2-9, can help appropriately select a λ for a particular cochlear implant recipient. For example, a recipient with good neural health at high frequencies can have a global λ value set that increases resolution at higher frequencies while decreasing resolution at lower frequencies with decreased neural health. On the other hand, a recipient with good neural health at low frequencies can have a global λ value set that increases resolution at lower frequencies while decreasing resolution at high frequencies. If respective local λs are used for each of the all-pass filter sections 1105a-w, the local λs can be set so that the recipient receives increased resolution for specific frequencies at which the recipient has good neural health.

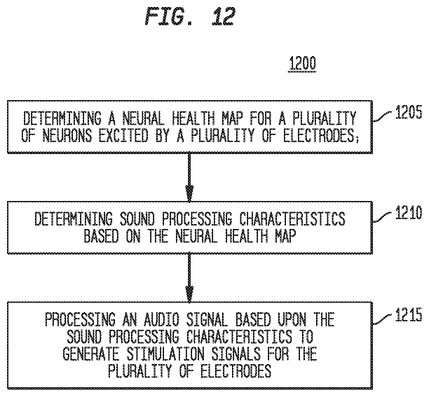

With reference now made to FIG. 12, depicted therein is a flowchart 1200 illustrating a generalized process flow for implementing the sound processing techniques of the present disclosure.

Flowchart 1200 begins in operation 1205 in which a neural health map is determined for a plurality of neurons excited by a plurality of electrodes (e.g., all or only a subset of electrodes implanted in a recipient). According to specific example embodiments, the determination of operation 1205 can be based upon the correlation of the stimulation necessary to evoke a response in an electrode with the distance between the electrode and the neurons. According to other example embodiments, the neural health map of operation 1205 can be determined through other techniques known to the skilled artisan. For example, neural health can be determined through techniques such as ECAP AGF slopes, the effect of changing IPG on the AGF curve, and SOE measures using panoramic PECAP. These methods can determine neuron health independent of electrode distance and may not require knowledge of electrode placement.

In operation 1210, sound processing characteristics are determined for a signal processor associated with the electrodes. The determination of the sound processing characteristics is based upon the neural health map. For example, operation 1210 can be embodied as the setting of a warping coefficient λ of a warped filter bank of the signal processor. The warping coefficient λ of the warped filter bank can be a global warping coefficient for the entirety of the warped filter bank or a local warping coefficient for an all-pass filter section of the warped filter bank.

Finally, in operation 1215, audio signals are processed based upon the sound processing characteristics to generate stimulation signals for the plurality of electrodes. For example, operation 1215 can be embodied as the processing of signals utilizing buffer block 1005, WDL block 1010, FFT block 1015, and CIC block 1020 of FIG. 10.

As previously described, the technology disclosed herein can be applied in any of a variety of circumstances and with a variety of different devices. Example devices that can benefit from technology disclosed herein are described in more detail in FIGS. 13-15, below. As described below, the operating parameters for the devices described with reference to FIGS. 13-15 can be configured using the neural health map techniques described above with reference to FIGS. 2-12. The techniques of the present disclosure can be applied to other medical devices, such as neurostimulators, cardiac pacemakers, cardiac defibrillators, sleep apnea management stimulators, seizure therapy stimulators, tinnitus management stimulators, and vestibular stimulation devices, as well as other medical devices that deliver stimulation to tissue. Further, technology described herein can also be applied to consumer devices. These different systems and devices can benefit from the technology described herein.

FIG. 13 is a functional block diagram of an implantable stimulator system 1300 that can benefit from the technologies described herein. The implantable stimulator system 1300 includes the wearable device 1301 acting as an external processor device and an implantable device 1303 acting as an implanted stimulator device. In examples, the implantable device 1303 is an implantable stimulator device configured to be implanted beneath a recipient's tissue (e.g., skin). In examples, the implantable device 1303 includes a biocompatible implantable housing 1302. Here, the wearable device 1301 is configured to transcutaneously couple with the implantable device 1303 via a wireless connection to provide additional functionality to the implantable device 30.

In the illustrated example, the wearable device 1301 includes one or more sensors 1312, a processor 1314, a transceiver 1318, and a power source 1348. The one or more sensors 1312 can be one or more units configured to produce data based on sensed activities. In an example where the stimulation system 1300 is an auditory prosthesis system, the one or more sensors 1312 include sound input sensors, such as a microphone, an electrical input for an FM hearing system, other components for receiving sound input, or combinations thereof. Where the stimulation system 1300 is a visual prosthesis system, the one or more sensors 1312 can include one or more cameras or other visual sensors. Where the stimulation system 1300 is a cardiac stimulator, the one or more sensors 1312 can include cardiac monitors. The processor 1314 can be a component (e.g., a central processing unit) configured to control stimulation provided by the implantable device 30. The stimulation can be controlled based on data from the sensor 1312, a stimulation schedule, or other data. Where the stimulation system 1300 is an auditory prosthesis, the processor 1314 can be configured to convert sound signals received from the sensor(s) 1312 (e.g., acting as a sound input unit) into signals 1351. The transceiver 1318 is configured to send the signals 1351 in the form of power signals, data signals, combinations thereof (e.g., by interleaving the signals), or other signals. The transceiver 1318 can also be configured to receive power or data. Stimulation signals can be generated by the processor 1314 and transmitted, using the transceiver 1318, to the implantable device 1303 for use in providing stimulation.

In the illustrated example, the implantable device 1303 includes a transceiver 1318, a power source 1348, and a medical instrument 1311 that includes an electronics module 1310 and a stimulator assembly 1330. The implantable device 1303 further includes a hermetically sealed, biocompatible implantable housing 1302 enclosing one or more of the components.

The electronics module 1310 can include one or more other components to provide medical device functionality. In many examples, the electronics module 1310 includes one or more components for receiving a signal and converting the signal into the stimulation signal 1315. The electronics module 1310 can further include a stimulator unit. The electronics module 1310 can generate or control delivery of the stimulation signals 1315 to the stimulator assembly 1330. In examples, the electronics module 1310 includes one or more processors (e.g., central processing units or microcontrollers) coupled to memory components (e.g., flash memory) storing instructions that when executed cause performance of an operation. In examples, the electronics module 1310 generates and monitors parameters associated with generating and delivering the stimulus (e.g., output voltage, output current, or line impedance). In examples, the electronics module 1310 generates a telemetry signal (e.g., a data signal) that includes telemetry data. The electronics module 1310 can send the telemetry signal to the wearable device 1301 or store the telemetry signal in memory for later use or retrieval.

The stimulator assembly 1330 can be a component configured to provide stimulation to target tissue. In the illustrated example, the stimulator assembly 1330 is an electrode assembly that includes an array of electrode contacts disposed on a lead. The lead can be disposed proximate tissue to be stimulated. Where the system 1300 is a cochlear implant system, the stimulator assembly 1330 can be inserted into the recipient's cochlea. The stimulator assembly 1330 can be configured to deliver stimulation signals 1315 (e.g., electrical stimulation signals) generated by the electronics module 1310 to the cochlea to cause the recipient to experience a hearing percept. In other examples, the stimulator assembly 1330 is a vibratory actuator disposed inside or outside of a housing of the implantable device 1303 and configured to generate vibrations. The vibratory actuator receives the stimulation signals 1315 and, based thereon, generates a mechanical output force in the form of vibrations. The actuator can deliver the vibrations to the skull of the recipient in a manner that produces motion or vibration of the recipient's skull, thereby causing a hearing percept by activating the hair cells in the recipient's cochlea via cochlea fluid motion.

The transceivers 1318 can be components configured to transcutaneously receive and/or transmit a signal 1351 (e.g., a power signal and/or a data signal). The transceiver 1318 can be a collection of one or more components that form part of a transcutaneous energy or data transfer system to transfer the signal 1351 between the wearable device 1301 and the implantable device 30. Various types of signal transfer, such as electromagnetic, capacitive, and inductive transfer, can be used to usably receive or transmit the signal 1351. The transceiver 1318 can include or be electrically connected to a coil 20.

As illustrated, the wearable device 1301 includes a coil 1308 for transcutaneous transfer of signals with coil 1320. As noted above, the transcutaneous transfer of signals between coil 1308 and the coil 1320 can include the transfer of power and/or data from the coil 1308 to the coil 1320 and/or the transfer of data from coil 1320 to the coil 1308. The power source 1348 can be one or more components configured to provide operational power to other components. The power source 1348 can be or include one or more rechargeable batteries. Power for the batteries can be received from a source and stored in the battery. The power can then be distributed to the other components as needed for operation.

As should be appreciated, while particular components are described in conjunction with FIG. 13, technology disclosed herein can be applied in any of a variety of circumstances. The above discussion is not meant to suggest that the disclosed techniques are only suitable for implementation within systems akin to that illustrated in and described with respect to FIG. 13. In general, additional configurations can be used to practice the methods and systems herein and/or some aspects described can be excluded without departing from the methods and systems disclosed herein.

FIG. 14 illustrates an example vestibular stimulator system 1402, with which embodiments presented herein can be implemented. As shown, the vestibular stimulator system 1402 comprises an implantable component (vestibular stimulator) 1412 and an external device/component 1404 (e.g., external processing device, battery charger, remote control, etc.). The external device 1404 comprises a transceiver unit 1460. As such, the external device 1404 is configured to transfer data (and potentially power) to the vestibular stimulator 1412,

The vestibular stimulator 1412 comprises an implant body (main module) 1434, a lead region 1436, and a stimulating assembly 1416, all configured to be implanted under the skin/tissue (tissue) 1415 of the recipient. The implant body 1434 generally comprises a hermetically-sealed housing 1438 in which RF interface circuitry, one or more rechargeable batteries, one or more processors, and a stimulator unit are disposed. The implant body 134 also includes an internal/implantable coil 1414 that is generally external to the housing 1438, but which is connected to the transceiver via a hermetic feedthrough (not shown).

The stimulating assembly 1416 comprises a plurality of electrodes 1444(1)-(3) disposed in a carrier member (e.g., a flexible silicone body). In this specific example, the stimulating assembly 1416 comprises three (3) stimulation electrodes, referred to as stimulation electrodes 1444(1), 1444(2), and 1444(3). The stimulation electrodes 1444(1), 1444(2), and 1444(3) function as an electrical interface for delivery of electrical stimulation signals to the recipient's vestibular system.

The stimulating assembly 1416 is configured such that a surgeon can implant the stimulating assembly adjacent the recipient's otolith organs via, for example, the recipient's oval window. It is to be appreciated that this specific embodiment with three stimulation electrodes is merely illustrative and that the techniques presented herein can be used with stimulating assemblies having different numbers of stimulation electrodes, stimulating assemblies having different lengths, etc.

In operation, the vestibular stimulator 1412, the external device 1404, and/or another external device, can be configured to implement the techniques presented herein. That is, the vestibular stimulator 1412, possibly in combination with the external device 1404 and/or another external device, can include an evoked biological response analysis system, as described elsewhere herein.

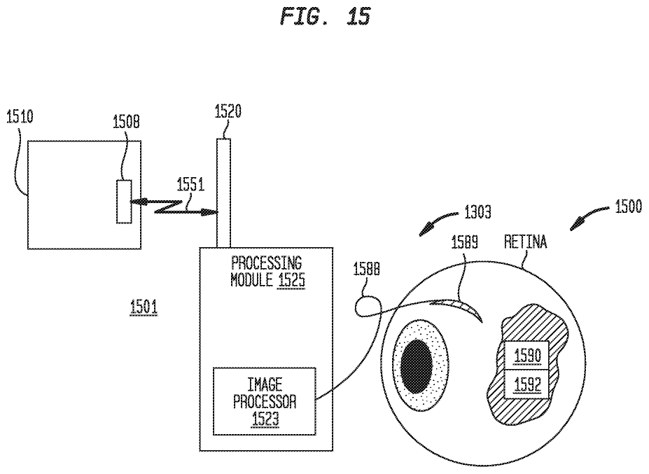

FIG. 15 illustrates a retinal prosthesis system 1501 that comprises an external device 1510 (which can correspond to the wearable device 1301) configured to communicate with a retinal prosthesis 1500 via signals 1551. The retinal prosthesis 1500 comprises an implanted processing module 1525 (e.g., which can correspond to the implantable device 1303) and a retinal prosthesis sensor-stimulator 1590 is positioned proximate the retina of a recipient. The external device 1510 and the processing module 1525 can communicate via coils 1508, 1520.

In an example, sensory inputs (e.g., photons entering the eye) are absorbed by a microelectronic array of the sensor-stimulator 1590 that is hybridized to a glass piece 1592 including, for example, an embedded array of microwires. The glass can have a curved surface that conforms to the inner radius of the retina. The sensor-stimulator 1590 can include a microelectronic imaging device that can be made of thin silicon containing integrated circuitry that convert the incident photons to an electronic charge.

The processing module 1525 includes an image processor 1523 that is in signal communication with the sensor-stimulator 1590 via, for example, a lead 1588 which extends through surgical incision 1589 formed in the eye wall. In other examples, processing module 1525 is in wireless communication with the sensor-stimulator 1590. The image processor 1523 processes the input into the sensor-stimulator 1590, and provides control signals back to the sensor-stimulator 1590 so the device can provide an output to the optic nerve. That said, in an alternate example, the processing is executed by a component proximate to, or integrated with, the sensor-stimulator 1590. The electric charge resulting from the conversion of the incident photons is converted to a proportional amount of electronic current which is input to a nearby retinal cell layer. The cells fire and a signal is sent to the optic nerve, thus inducing a sight perception.

The processing module 1525 can be implanted in the recipient and function by communicating with the external device 1510, such as a behind-the-ear unit, a pair of eyeglasses, etc. The external device 1510 can include an external light/image capture device (e.g., located in/on a behind-the-ear device or a pair of glasses, etc.), while, as noted above, in some examples, the sensor-stimulator 1590 captures light/images, which sensor-stimulator is implanted in the recipient.

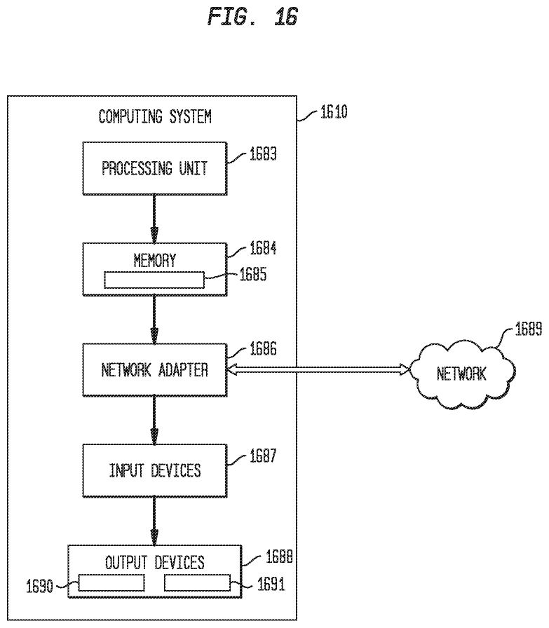

FIG. 16 is a function block diagram of a computing device 1610, configured to implement aspects of the techniques presented (e.g., implement aspects of one or more of the methods described herein). The computing device 1610, is, for example, a personal computer, server computer, hand-held device, laptop device, multiprocessor system, microprocessor-based system, programmable consumer electronic (e.g., smart phone), network PC, minicomputer, mainframe computer, tablet, remote control unit, distributed computing environment that include any of the above systems or devices, and the like. The computing device 1610 can be a single virtual or physical device operating in a networked environment over communication links to one or more remote devices, such as an implantable medical device or implantable medical device system.

In its most basic configuration, computing device 1610 includes at least one processing unit 1683 and memory 1684. The processing unit 1683 includes one or more hardware or software processors (e.g., Central Processing Units) that can obtain and execute instructions. The processing unit 1683 can communicate with and control the performance of other components of the computing system 1610.

The memory 1684 is one or more software or hardware-based computer-readable storage media operable to store information accessible by the processing unit 1683. The memory 1684 can store, among other things, instructions executable by the processing unit 1683 to implement applications or cause performance of operations described herein, as well as other data. The memory 1684 can be volatile memory (e.g., RAM), non-volatile memory (e.g., ROM), or combinations thereof. The memory 884 can include transitory memory or non-transitory memory. The memory 1684 can also include one or more removable or non-removable storage devices. In examples, the memory 1684 can include RAM, ROM, EEPROM (Electronically-Erasable Programmable Read-Only Memory), flash memory, optical disc storage, magnetic storage, solid state storage, or any other memory media usable to store information for later access. In examples, the memory 1684 encompasses a modulated data signal (e.g., a signal that has one or more of its characteristics set or changed in such a manner as to encode information in the signal), such as a carrier wave or other transport mechanism and includes any information delivery media. By way of example, and not limitation, the memory 1684 can include wired media such as a wired network or direct-wired connection, and wireless media such as acoustic, RF, infrared and other wireless media or combinations thereof. In certain embodiments, the memory 1684 comprises neural map logic 1685 that, when executed, enables the processing unit 1683 to perform aspects of the techniques presented.

In the illustrated example, the system 1610 further includes a network adapter 1686, one or more input devices 1687, and one or more output devices 1688. The system 1610 can include other components, such as a system bus, component interfaces, a graphics system, a power source (e.g., a battery), among other components.

The network adapter 1686 is a component of the computing system 1610 that provides network access (e.g., access to at least one network 1689). The network adapter 1686 can provide wired or wireless network access and can support one or more of a variety of communication technologies and protocols, such as ETHERNET, cellular, BLUETOOTH, near-field communication, and RF (Radiofrequency), among others. The network adapter 1686 can include one or more antennas and associated components configured for wireless communication according to one or more wireless communication technologies and protocols.

The one or more input devices 1687 are devices over which the computing system 1610 receives input from a user. The one or more input devices 1687 can include physically-actuatable user-interface elements (e.g., buttons, switches, or dials), touch screens, keyboards, mice, pens, and voice input devices, among others input devices.

The one or more output devices 1688 are devices by which the computing system 1610 is able to provide output to a user. The output devices 1688 can include, a display 1690 and one or more speakers 1691, among other output devices.

In one implementation, the network adapter 1686 (or another type of input/interface) can be configured to send instructions to, or receive data from an implantable device, such as a cochlear implant, or from a component in communication with an implantable device (e.g., communicate directly with the cochlear implant or communicate with a sound processor inductively coupled to a cochlear implant). The processing unit 1683 could execute the neural map logic 1685 to perform one or more of the calculations, correlations, and other operations described herein to, for example, generate a neural map. The output devices 1688 can be used to provide results to a user.

It is to be appreciated that the arrangement for computing system 1610 shown in FIG. 1E is merely illustrative and that aspects of the techniques presented herein can be implemented at a number of different types of systems/devices. For example, the computing system 1610 could be a laptop computer, tablet computer, mobile phone, surgical system, etc.

As should be appreciated, while particular uses of the technology have been illustrated and discussed above, the disclosed technology can be used with a variety of devices in accordance with many examples of the technology. The above discussion is not meant to suggest that the disclosed technology is only suitable for implementation within systems akin to that illustrated in the figures. In general, additional configurations can be used to practice the processes and systems herein and/or some aspects described can be excluded without departing from the processes and systems disclosed herein.

This disclosure described some aspects of the present technology with reference to the accompanying drawings, in which only some of the possible aspects were shown. Other aspects can, however, be embodied in many different forms and should not be construed as limited to the aspects set forth herein. Rather, these aspects were provided so that this disclosure was thorough and complete and fully conveyed the scope of the possible aspects to those skilled in the art.

As should be appreciated, the various aspects (e.g., portions, components, etc.) described with respect to the figures herein are not intended to limit the systems and processes to the particular aspects described. Accordingly, additional configurations can be used to practice the methods and systems herein and/or some aspects described can be excluded without departing from the methods and systems disclosed herein.

According to certain aspects, systems and non-transitory computer readable storage media are provided. The systems are configured with hardware configured to execute operations analogous to the methods of the present disclosure. The one or more non-transitory computer readable storage media comprise instructions that, when executed by one or more processors, cause the one or more processors to execute operations analogous to the methods of the present disclosure.

Similarly, where steps of a process are disclosed, those steps are described for purposes of illustrating the present methods and systems and are not intended to limit the disclosure to a particular sequence of steps. For example, the steps can be performed in differing order, two or more steps can be performed concurrently, additional steps can be performed, and disclosed steps can be excluded without departing from the present disclosure. Further, the disclosed processes can be repeated.

Although specific aspects were described herein, the scope of the technology is not limited to those specific aspects. One skilled in the art will recognize other aspects or improvements that are within the scope of the present technology. Therefore, the specific structure, acts, or media are disclosed only as illustrative aspects. The scope of the technology is defined by the following claims and any equivalents therein.

It is also to be appreciated that the embodiments presented herein are not mutually exclusive and that the various embodiments can be combined with another in any of a number of different manners.

1. A method, comprising:
determining, for each electrode of a plurality of electrodes, a distance between each electrode of the plurality of electrodes and one or more neurons;
determining a stimulation threshold for each electrode of the plurality of electrodes to evoke a response in the one or more neurons;
correlating the stimulation threshold for each electrode of the plurality of electrodes with the distance between each electrode of the plurality of electrodes and the one or more neurons; and
generating a neural health map for the one or more neurons based upon the correlating the stimulation threshold for each electrode of the plurality of electrodes with the distance between each electrode of the plurality of electrodes and the one or more neurons.

2. The method of claim 1, wherein determining the stimulation threshold for each electrode of the plurality of electrodes to evoke a response in the one or more neurons comprises:
determining an Electrically-evoked Compound Action Potential (ECAP) threshold for each of the plurality of electrodes.

3. The method of claim 1, wherein generating a neural health map for the plurality of electrodes based upon the correlating comprises:
comparing a distance between a first electrode of the plurality of electrodes and a first region associated with the one or more neurons with the stimulation threshold for the first electrode of the plurality of electrodes; and
determining a health level of the first region from the comparing of the distance between the first electrode of the plurality of electrodes and the first region associated with the one or more neurons with the stimulation threshold for the first electrode.

4. The method of claim 1, wherein the plurality of electrodes comprises a plurality of electrodes for a cochlear implant.

5. The method of claim 4, wherein the one or more neurons comprise neurons arranged within a cochlea of a recipient of the cochlear implant.

6. The method of claim 1, wherein the plurality of electrodes comprises a plurality of electrodes for an implantable stimulator system.

7. The method of claim 1, wherein the plurality of electrodes comprises a plurality of electrodes for a vestibular stimulator system.

8. The method of claim 1, wherein the plurality of electrodes comprises a plurality of electrodes for a retinal prosthesis system.

9. The method of claim 1, wherein determining the distance between each electrode of the plurality of electrodes and one more neurons comprises imaging each electrode using Computed Tomography imaging.

10. The method of any one of claim 1, wherein determining the distance between each electrode of the plurality of electrodes and one more neurons comprises imaging each electrode using magnetic resonance imaging.

11. The method of claim 1, wherein the plurality of electrodes are a subset of electrodes implanted in a recipient.

12. (canceled)

13. The method of claim 1, wherein the plurality of electrodes are all electrodes implanted in a recipient.

14-25. (canceled)

26. One or more non-transitory computer readable storage media comprising instructions that, when executed by a processor, cause the processor to:
obtain for each electrode of a plurality of electrodes, a distance between each electrode of the plurality of electrodes and one or more neurons;
obtain a stimulation threshold for each electrode of the plurality of electrodes to evoke a response in the one or more neurons;
correlate the stimulation threshold for each electrode of the plurality of electrodes with the distance between each electrode of the plurality of electrodes and the one or more neurons; and
configure a hearing device based on the correlation of the stimulation threshold for each electrode of the plurality of electrodes with the distance between each electrode of the plurality of electrodes and the one or more neurons.

27. The one or more non-transitory computer readable storage media of claim 26, wherein the instructions operable to obtain the stimulation threshold for each electrode of the plurality of electrodes to evoke a response in the one or more neurons comprise instructions operable to:
determine an Electrically-evoked Compound Action Potential threshold for each of the plurality of electrodes.

28. The one or more non-transitory computer readable storage media of claim 26, wherein the instructions operable to correlate the stimulation threshold for each electrode of the plurality of electrodes with the distance between each electrode of the plurality of electrodes and the one or more neurons comprise instructions operable to:
compare a distance between a first electrode of the plurality of electrodes and a first region associated with the one or more neurons with the stimulation threshold for the first electrode of the plurality of electrodes; and
determine a health level of the first region from the comparing of the distance between the first electrode of the plurality of electrodes and the first region associated with the one or more neurons with the stimulation threshold for the first electrode.

29. The one or more non-transitory computer readable storage media of claim 26, wherein the instructions operable to obtain the distance between each electrode of the plurality of electrodes and one or more neurons comprise instructions operable to:
determine the distance between each electrode of the plurality of electrodes and one more neurons based on imaging data.

30. The one or more non-transitory computer readable storage media of claim 29, wherein the imaging data comprises Computed Tomography imaging data.

31. The one or more non-transitory computer readable storage media of claim 29, wherein the imaging data comprises magnetic resonance imaging data.

32. The one or more non-transitory computer readable storage media of claim 29, wherein the instructions operable to configure the hearing device based on the correlation of the stimulation threshold for each electrode of the plurality of electrodes with the distance between each electrode of the plurality of electrodes and the one or more neurons, comprise instructions operable to:
determine stimulation characteristics for use by the hearing device in stimulating each of a plurality of neuron regions.

33. The one or more non-transitory computer readable storage media of claim 32, further instructions operable to:
determine a trans-impedance matrix for the plurality of electrodes.

34. The one or more non-transitory computer readable storage media of claim 32, wherein the instructions operable to determine stimulation characteristics for use by hearing device in stimulating each of a plurality of neuron regions comprises instructions operable to:
determine a degree of focusing for focused multipolar stimulation for use by the hearing device in stimulating each of a plurality of neuron regions.

35. A system, comprising:
a memory; and
one or more processors configured to:
determine a neural health map for a plurality of neurons excited by a plurality of electrodes of an implantable medical device;
determine stimulation characteristics for the plurality of neurons based on the neural health map; and
program the implantable medical device to stimulate the plurality of neurons via the plurality of electrodes based upon the stimulation characteristics.

36. The system of claim 35, wherein determining the neural health map comprises determining neural health for a plurality of regions associated with the plurality of neurons.

37. The system of claim 36, wherein determining the stimulation characteristics comprises determining stimulation characteristics for each of the plurality of regions.

38. The system of claim 35, wherein to determine the neural health map for the plurality of neurons, the one or more processors are configured to:
determine, for each electrode of the plurality of electrodes, a distance between each electrode of the plurality of electrodes and one or more neurons of the plurality of neurons;
determine a stimulation threshold for each electrode of the plurality of electrodes to evoke a response in the one or more neurons;
correlate the stimulation threshold for each electrode of the plurality of electrodes with the distance between each electrode of the plurality of electrodes and the one or more neurons; and
generate the neural health map for the plurality of neurons by the correlating the stimulation threshold for each electrode of the plurality of electrodes with the distance between each electrode of the plurality of electrodes and the one or more neurons.

39. The system of claim 35, wherein the one or more processors are configured to:
determine a trans-impedance matrix for the plurality of electrodes.

40. The system of claim 35 wherein to determine the stimulation characteristics, the one or more processors are configured to:
determine a degree of focusing for focused multipolar stimulation of the plurality of neurons.
